(19) United States
(12) Patent Application Publication
(10) Pub. No.: US 2021/0163926 A1
(43) Pub. Date: Jun. 3, 2021

(54) VERSATILE AMPLICON SINGLE-CELL DROPLET SEQUENCING-BASED SHOTGUN SCREENING PLATFORM TO ACCELERATE FUNCTIONAL GENOMICS

(71) Applicant: ARIZONA BOARD OF REGENTS ON BEHALF OF ARIZONA STATE UNIVERSITY, Scottsdale, AZ (US)

(72) Inventors: Joshua LABAER, Chandler, AZ (US); Jin PARK, Phoenix, AZ (US)

(73) Assignee: ARIZONA BOARD OF REGENTS ON BEHALF OF ARIZONA STATE UNIVERSITY, Scottsdale, AZ (US)

(21) Appl. No.: 16/954,201

(22) PCT Filed: Jan. 3, 2019
(86) PCT No.: PCT/US2019/012210

§ 371 Date: Jul. 2, 2020

Related U.S. Application Data
(60) Provisional application No. 62/613,644, filed on Jan. 4, 2018.

(51) Int. Cl. C12N 15/10

(52) U.S. Cl. CPC C12N 15/1068

(57) ABSTRACT

Disclosed is a method of functional genomics determination including transducing a cell population with a set of nucleic acid molecules including a pooled library of genomic perturbagens to integrate multiple perturbagen cassettes into the genome. A phenotype of individual cells is determined and single cells of the population with targeted phenotypes are individually sorted into a set of compartments. Each compartment includes a forward primer with a nucleic acid sequence (NAS) that specifically binds a common nucleic acid sequence on the nucleic acid molecules and a compartment (cell)-specific nucleic acid barcode. Also included is a reverse primer with a NAS that specifically binds a common NAS on the nucleic acid molecules comprising a pooled library of genomic perturbagens. The genome-integrated perturbagen cassettes are create amplicons which are pooled and sequences determined. This method can be applied to other genome-level single-cell applications—immune receptor profiling, targeted DNA/RNA sequencing, and metagenomics.

### CROSS REFERENCE TO RELATED APPLICATION

This application claims the benefit of U.S. Provisional Application No. 62/613,644, filed on Jan. 4, 2018 which is incorporated herein by reference in its entirety.

### FIELD

This disclosure relates to functional genomics, and, in particular, to the methods and compositions for determining the effect of multiplex genetic perturbations introduced into a cell population.

### BACKGROUND

Fast expanding genomic sequencing data have revealed a massive landscape of thousands of somatic mutations in cancer, which represent both driver mutations and by-standing passenger mutations (Pon and Marra, Annu Rev Pathol. 2015; 10:25-50). Adding more complexity, the recent development of single cell sequencing technology (Gawad, Koh, and Quake, Nat Rev Genet. 2016 March; 17(3):175-88; Baslan and Hicks, Nat Rev Cancer. 2017 Aug. 24; 17(9):557-569) has led to identification of heterogeneous clonal populations within a single tumor that carry unique combinations of multiple driver and passenger mutations. Elucidating the functionally-relevant combinations among the milieu of mutations is key for not only understanding the clonal development of cancer but also for developing personalized and targeted therapies (Wang et al., Semin Cancer Biol. 2017 February; 42:44-51); thus, enormous efforts have been put into functional genomics screens. These are typically based on pooled genome-scale libraries of perturbagens, such as shRNA and CRISPR/Cas9 gRNAs (Rauscher et al., Nucleic Acids Res. 2017 Jan. 4; 45(D1): D679-D686) However, given that metastatic tumor cells typically contain more than 3 coexisting functional driver mutations (Domcke et al., Nat Commun. 2013; 4:2126) conventional pool-based screening approaches that only test one perturbagen in a cell at a time have clear drawbacks in identifying functionally important mutation combinations.

A typical genome-wide screening approach involves a low MOI (multiplicity of infection) transduction of a pooled lentiviral library to introduce only a single perturbagen into a single cell, followed by selection of cells with desired phenotypes, PCR amplification of integrated constructs with universal primers, and bulk next-generation sequencing (Shalem, Nat Rev Genet. 2015 May; 16(5):299-311). Therefore, to identify coexisting combinatorial perturbations that induce targeted phenotypes, multiple rounds of successive clonal expansion and screens (i.e., “stepwise clonal screen”) are required (FIG. 1, left panel), which is extremely time/labor consuming and difficult to scale-up to accommodate the complexity of genome-wide combinatorial perturbations. More importantly, the conventional stepwise screen approach can suffer from a low discovery rate, as only a handful of the founding mutations would be selected and carried over to the next screening rounds. Moreover, mutations that work singly may not be the same mutations that work in combinations. Therefore, improved methods and systems are needed to overcome these deficiencies.

### BRIEF DESCRIPTION OF DRAWINGS

FIG. 1 is a flow-chart illustrating two screening strategies for identification of combinatorial gRNAs that promote cell invasion. Key differences between two approaches are highlighted.

FIG. 2 is a schematic diagram on amplification of gRNA cassettes in single cells by Amp-Drop-Seq.

FIG. 3 is a schematic illustration of an exemplary Amp-Drop-Seq procedure. For illustrative purposes, the linker portion is simplified, and only the PCR reaction starting by reverse primers is shown.

FIG. 4 is a schematic illustration of an exemplary Amp-Drop-Seq procedure customized for reading gRNA cassettes from genomic DNA in parallel with mRNA levels of genes of interest. For illustrative purposes, the linker portion is simplified.

### DETAILED DESCRIPTION

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

The singular forms “a,” “an,” and “the” refer to one or more than one, unless the context clearly dictates otherwise. For example, the term “comprising” includes single or plural forms and is considered equivalent to the phrase “comprising at least one.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

The term “contact” along with its derivatives, may be used. It should be understood that these terms are not intended as synonyms for each other. Rather, in particular embodiments, “contacted” means that two or more elements are in direct physical contact. However, “contacted” can also mean that two or more elements are not in direct contact with each other, but yet still cooperate or interact with each other.

In order to facilitate review of the various embodiments of this disclosure, the following explanations of specific terms are provided:

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR), real-time PCR (rt FOR); real-time reverse transcriptase PCR (rt RT-PCR), nested FOR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Binding or stable binding: An association between two substances or molecules, such as the hybridization of one nucleic acid molecule to another or itself, the association of an antibody with a peptide, or the association of a protein with another protein or nucleic acid molecule.

Capture moieties: Molecules or other substances that when attached to another molecule, such as a nucleic acid, allow for the capture of the targeting probe through interactions of the capture moiety and something that the capture moiety binds to, such as a particular surface and/or molecule, such as a specific binding molecule that is capable of specifically binding to the capture moiety. In specific examples, a capture moiety is biotin and a capture moiety specific binding agent is avidin or streptavidin.

Compartment: A discrete volume or discrete space, such as a container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a cell and a indexable nucleic acid identifier (for example nucleic acid barcode or nucleic acid molecule including a nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in our through the discrete volume, while other material, such as cells, maybe maintained in the discrete volume or space. Typically, a discrete volume will include a fluid medium, (for example, an aqueous solution, an oil, a buffer, and/or a media capable of supporting cell growth). Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion.

Conditions sufficient to detect: Any environment that permits the detection of the desired activity, for example, that permits detection and/or quantification of a nucleic acid, such as a genomic perturbagens, a nucleic acid barcode, a transcription product, and/or amplification product thereof.

Control: A reference standard. A control can be a known value or range of values indicative of basal levels or amounts or present in a tissue or a cell or populations thereof (such as a normal non-cancerous cell). A control can also be a cellular or tissue control, for example a tissue from a non-diseased state and/or exposed to different environmental conditions. A difference between a test sample and a control can be an increase or conversely a decrease. The difference can be a qualitative difference or a quantitative difference, for example a statistically significant difference.

Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between nucleic acid oligonucleotide and a solid or semisolid substrate, such a bead, for example a hydrogel bead.

Detect: To determine if an agent (such as a signal or particular nucleic acid, such a nucleic acid barcode, or a genomic perturbagens) is present or absent. In some examples, this can further include quantification in a sample, or a fraction of a sample, such as a particular cell or cells.

Detectable label: A compound or composition that is conjugated directly or indirectly to another molecule to facilitate detection of that molecule. Specific, non-limiting examples of labels include fluorescent tags, enzymatic linkages, and radioactive isotopes. In some examples, a label is attached to an antibody or nucleic acid to facilitate detection of the molecule antibody or nucleic acid specifically binds. In specific examples, a detectable label comprises a nucleic acid barcode.

DNA sequencing: The process of determining the nucleotide order of a given DNA molecule. Generally, the sequencing can be performed using automated Sanger sequencing (AB 13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®). In some embodiments, the identity of a nucleic acid is determined by DNA or RNA sequencing. Generally, the sequencing can be performed using automated Sanger sequencing (ABI 3730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing.

In some embodiments, DNA sequencing is performed using a chain termination method developed by Frederick Sanger, and thus termed “Sanger based sequencing” or “SBS.” This technique uses sequence-specific termination of a DNA synthesis reaction using modified nucleotide substrates. Extension is initiated at a specific site on the template DNA by using a short oligonucleotide primer complementary to the template at that region. The oligonucleotide primer is extended using DNA polymerase in the presence of the four deoxynucleotide bases (DNA building blocks), along with a low concentration of a chain terminating nucleotide (most commonly a di-deoxynucleotide). Limited incorporation of the chain terminating nucleotide by the DNA polymerase results in a series of related DNA fragments that are terminated only at positions where that particular nucleotide is present. The fragments are then size-separated by electrophoresis a polyacrylamide gel, or in a narrow glass tube (capillary) filled with a viscous polymer. An alternative to using a labeled primer is to use labeled terminators instead; this method is commonly called “dye terminator sequencing.”

“Pyrosequencing” is an array based method, which has been commercialized by 454 Life Sciences. In some embodiments of the array-based methods, single-stranded DNA is annealed to beads and amplified via Em FOR®. These DNA-bound beads are then placed into wells on a fiber-optic chip along with enzymes that produce light in the presence of ATP. When free nucleotides are washed over this chip, light is produced as the PCR amplification occurs and ATP is generated when nucleotides join with their complementary base pairs. Addition of one (or more) nucleotide(s) results in a reaction that generates a light signal that is recorded, such as by the charge coupled device (CCD) camera, within the instrument. The signal strength is proportional to the number of nucleotides, for example, homopolymer stretches, incorporated in a single nucleotide flow.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization.

Isolated: An “isolated” biological component (such a nucleic acid) has been substantially separated or purified away from other biological components in the cell of the organism in which the component naturally occurs, for example, extra-chromatin DNA and RNA, proteins and organelles. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

Multiplicity of Infection (MOI): A term used herein to reference the ratio of agents, such as perturbagen, to infection targets (for example, cell). For example, when referring to a group of cells contacted with a perturbagen, the multiplicity of infection or MOI is the ratio of the number of perturbagens capable of modification of a host cell to the number of target cells present. Herein, a low MOI range is referring to below 0.5, where >75% of transduced cells are transduced with only a single gRNA based on the predicted Poisson distribution. A high MOI is above 3.0, where >85% of transduced cells are transduced with 2 or more gRNAs. A midrange MOI is referring to one between 0.5 and 3.0, which can generate a diverse population of cells with 1 to more than 3 gRNAs.

Nucleic acid (molecule or sequence): A deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major building blocks for polymeric nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major building blocks for polymeric nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

In some examples, nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others. Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Nucleic acid barcode, barcode, unique molecular identifier, or UMI: A short sequence of nucleotides (for example, DNA, RNA, or combinations thereof) that is used as an identifier for an associated molecule, such as a target molecule and/or target nucleic acid, for example cell type or phenotype, or a particular genomic perturbagens. A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes and/or UMIs can be attached, or “tagged,” to a target molecule and/or target nucleic acid. This attachment can be direct (for example, covalent or noncovalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify a target as being from a particular compartment (for example a discrete volume), having a particular physical property (for example, affinity, length, sequence, etc.), or having been subject to certain treatment conditions or genomic perturbagens. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discrete volume-, physical property-, or treatment condition-specific) nucleic acid barcodes.

Perturbagen: Any modality, such as an agent or collection of agents, that can be administered to to determine the biological response to the perturbagen. In an embodiment, a perturbagen is a genetic alteration, for example, as implemented by CRISPR genetics. In an embodiment, perturbagen is a genome-integrated perturbagen cassette.

Primers: Short nucleic acid molecules, such as a DNA oligonucleotide, for example sequences of at least 15 nucleotides, which can be annealed to a complementary nucleic acid molecule by nucleic acid hybridization to form a hybrid between the primer and the nucleic acid strand. A primer can be extended along the nucleic acid molecule by a polymerase enzyme. Therefore, primers can be used to amplify a nucleic acid molecule, wherein the sequence of the primer is specific for the nucleic acid molecule, for example so that the primer will hybridize to the nucleic acid molecule under very high stringency hybridization conditions. The specificity of a primer increases with its length. Thus, for example, a primer that includes 30 consecutive nucleotides will anneal to a sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides.

In particular examples, a primer is at least 15 nucleotides in length, such as at least 15 contiguous nucleotides complementary to a nucleic acid molecule. Particular lengths of primers that can be used to practice the methods of the present disclosure, include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the nucleic acid molecule to be amplified, such as a primer of 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, Mass.).

Methods for preparing and using primers are described in, for example, Sambrook et al. (1989) Molecular Cloning: A Laboratory Manual, Cold Spring Harbor, New York; Ausubel et al. (1987) Current Protocols in Molecular Biology, Greene Publ. Assoc. & Wiley-lntersciences. In one example, a primer includes a label.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5: 151-3, 1989; Corpet et al, Nuc. Acids Res. 16: 10881-90, 1988; Huang et al. Computer Appls. in the Biosciences 8, 155-65, 1992; and Pearson et al, Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al, J. Mol. Biol. 215:403-10, 1990) is available from several sources, including the National Center for Biological Information (NCBI, National Library of Medicine, Building 38 A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166+1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15+20* 100=75).

Specific Binding Agent: An agent that binds substantially or preferentially only to a defined target such as a polypeptide protein, enzyme, polysaccharide, oligonucleotide, DNA, RNA, recombinant vector or a small molecule.

A nucleic acid-specific binding agent binds substantially only to the defined nucleic acid, such as RNA, or to a specific region within the nucleic acid.

Support: A solid or semisolid substrate to which something can be attached, such as a oligonucleotide including a nucleic acid barcode. The attachment can be a removable attachment. Non-limiting examples of a support useful in the methods of the disclosure include a hydrogel, cell, bead, column, filter, slide surface, or interior wall of a compartment, such as a well in a microtiter plate, or vessel. In certain embodiments, the support is a hydrogel (such as a hydrogel bead) to which one or more nucleic acid oligonucleotides including a is coupled nucleic acid barcode. A nucleic acid oligonucleotides including a coupled nucleic acid barcode reversibly coupled to a support can be detached from the support, for example photo and or enzymatic cleavage of a cleavage site. A support may be present in a compartment as set forth herein. In certain embodiments, the support is a hydrogel bead present in an emulsion droplet.

Suitable methods and materials for the practice or testing of this disclosure are described herein. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

All publications, patent applications, patents, and other references mentioned herein are incorporated by reference in their entirety as available. In case of conflict, the present specification, including explanations of terms, will control. In addition, the materials, methods, and examples are illustrative only and not intended to be limiting.

### Introduction

Aggressive cancers often have up to hundreds of somatic mutations including a few known cancer driver mutations, and it is believed that only a small fraction of mutations (or “co-driver” mutations) contribute to cancer progression in collaboration with driver mutations. Although massive research efforts have been made to identify co-drivers that work together, either by genome-wide functional genomics screens or genomics data-derived targeted studies, the identities of functional and clinically important co-drivers is still largely unknown, mainly due to the extreme heterogeneity and diversity of possible mutational combinations that need to be screened and tested. By using the conventional screening approach of using pooled CRISPR or shRNA libraries, only one perturbagen can be introduced and tested in each cell for phenotypic effects, as coexistence information of multiple perturbagens within a single cell cannot be decomposed from the bulk sequencing data. Although an array of recently developed microfluidics-based single-cell sequencing technologies holds promise as a potential solution, currently available commercial and non-commercial platforms are optimized for whole genome/exome/transcriptome sequencing applications, but not for identification of exogenous perturbagens residing in a single cell. As the genome-integrated perturbagen sequences are extremely small (typically thousands of bases) when compared to the entire human genome with 3 billion bases, current platforms have severe limitations in obtaining enough sequencing depths for the targeted perturbagen sequences, and thus would be very costly to achieve enough statistical power for identification of positive hits. Therefore, disclosed herein is a cell-based screening pipeline based on a single-cell droplet sequencing platform called Amp-Drop-Seq that is specifically designed to amplify and detect multiple gRNAs or shRNAs at the single cell level for functional genomics screens. By allowing the “shotgun screen” approach of transducing and testing multiple perturbagens in parallel, this screening pipeline will provide significant advantages over the conventional screening methods. First, it can unveil novel mutational combinations that contribute to cancer progression only as a group but not as single mutations. These combinations cannot be identified by sequentially adding and testing multiple mutations. Secondly, Amp-Drop-Seq can greatly accelerate target discovery process by eliminating the elaborate and time-consuming process of multiple rounds of screens with a single perturbagen and clonal expansion. Furthermore, this highly versatile platform can be adapted for many other genome applications such as single-cell targeted exome sequencing, RNA sequencing, metagenomics, metatranscriptomics, as well as molecular profiling of immune cell populations. In certain implemented embodiments, the methods disclosed herein are used to identify genes that are expressed together in the same cell, for example, pairs or groups of genes that are co-expressed in diseased cells like cancer cells, co-expressed receptor proteins such as separate chains of T cell receptors, other subunits of cell surface receptors, etc. and for the determination of co-expression of proteins with specific alleles in situations of allele suppression. (e.g., X inactivation).

### Overview Of Several Embodiments

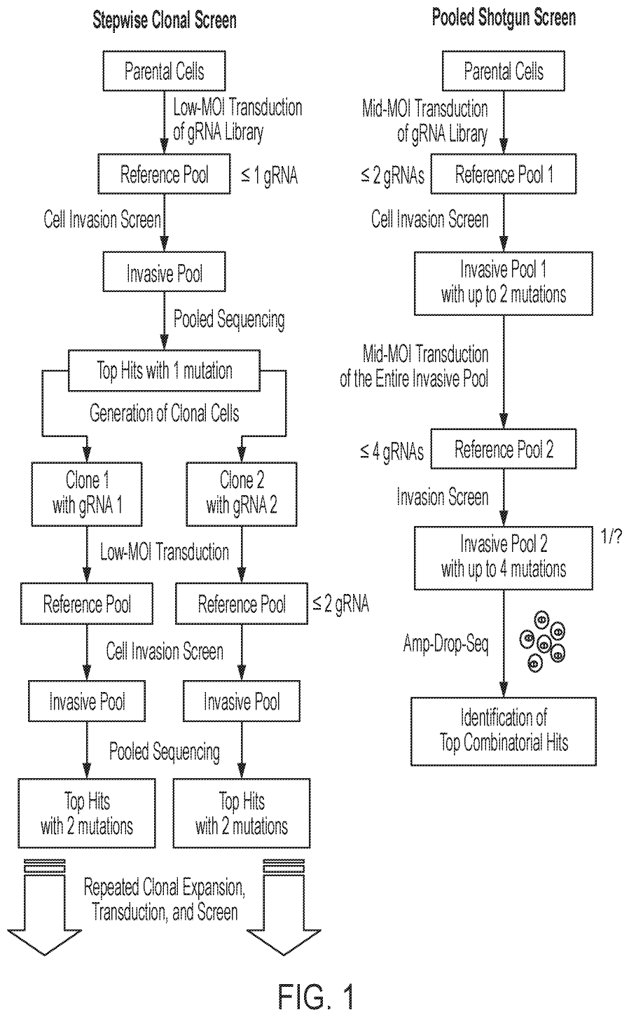

A conceptually rational and simpler alternative approach to overcome the limitations of current methods is disclosed herein. As illustrated in FIG. 1, right panel, an exemplary “pooled shotgun screen” is disclosed, where more than one CRISPR gRNA or shRNA are introduced at once or serially into a cell by transduction at higher MOI with the subsequent high-throughput assessment of which perturbations co-exist in individual cells of the “selected” population with targeted phenotypes. The current methods of bulk sequencing have a critical limitation in that co-occurrence information cannot be decomposed computationally from the sequencing results. Therefore, disclosed herein is a novel single-cell amplicon sequencing platform based on the state-of-the-art barcoded droplet sequencing technology (Amp-Drop-Seq, hereafter), which will greatly accelerate the discovery process of pathologically important mutational combinations among the ever-growing compendium of somatic mutations for development of targeted therapies for aggressive cancers.

Widely used droplet- or microwell-based single-cell platforms such as Chromium (10X Genomics), C1 (Fluidigm), Drop-Seq (Macosko, E.Z., et al., Highly Parallel Genome-wide Expression Profiling of Individual Cells Using Nanoliter Droplets. Cell, 2015. 161(5): p. 1202-14), and inDrop (Klein, A.M., et al., Droplet barcoding for single-cell transcriptomics applied to embryonic stem cells. Cell, 2015. 161(5): p. 1187-201), Zilionis, R., et al., Single-cell barcoding and sequencing using droplet microfluidics. Nat Protoc, 2017. 12(1): p. 44-73) are highly optimized for genome-scale DNA-Seq or RNA-Seq by utilizing tagmentation or by poly-A-based total mRNA capture. However, despite offering a significant benefit over bulk sequencing in global molecular profiling at the single-cell level, these platforms are not suitable for library-based functional genomics screening applications as they do not support targeted amplicon generation in droplets and/or have limited throughput. This means that the researcher ends up with mostly redundant information about the entire genome, which markedly dilutes out the specific information about the genes or perturbagens that were tested. Therefore, the strength of well-established droplet sequencing and microwell PCR technologies are combined in the proposed single-cell amplicon-targeted droplet sequencing platform, Amp-Drop-Seq, based on single-cell capture in droplets with barcoded beads and encapsulated PCR with universal primers. This novel platform is uniquely and specifically designed to support and accelerate functional genomics screening applications by allowing introduction and testing multiple in each cell providing unprecedented capability and throughput.

As this platform can provide information on co-occurrence of multiple genome-integrated perturbagens in a single cell, unlike conventional screening methods of testing the effect of only one perturbagen per cell, multiple perturbations can be simultaneously introduced to a cell, and the combinatorial effects can be screened in parallel. This would be the first tool of its kind. Furthermore, as only the amplicons, not the whole genome/transcriptome as in other droplet sequencing platforms, are sequenced, it can handle the complexity of combinatorial perturbagens and millions of cells with the existing next-generation sequencers. Taken together, this innovative technology will not only speed up the progress of target discovery but also unveil the previously unknown functional crosstalk between multiple genes and mutations.

Further, with simple modifications, this platform can be applied to many other genome-level applications. For example, by multiplexing the primer sets, this platform can be used for targeted single-cell exome sequencing for large-scale studies on tumor heterogeneity and clonal evolution in millions of cells, determining which genome alterations occur together in individual cells. Alternatively, to profile expression of genes of interest in large number of cells, targeted single-cell RNA-Seq can be done by combining reverse transcription reactions and multiplexed amplification of specific transcripts.

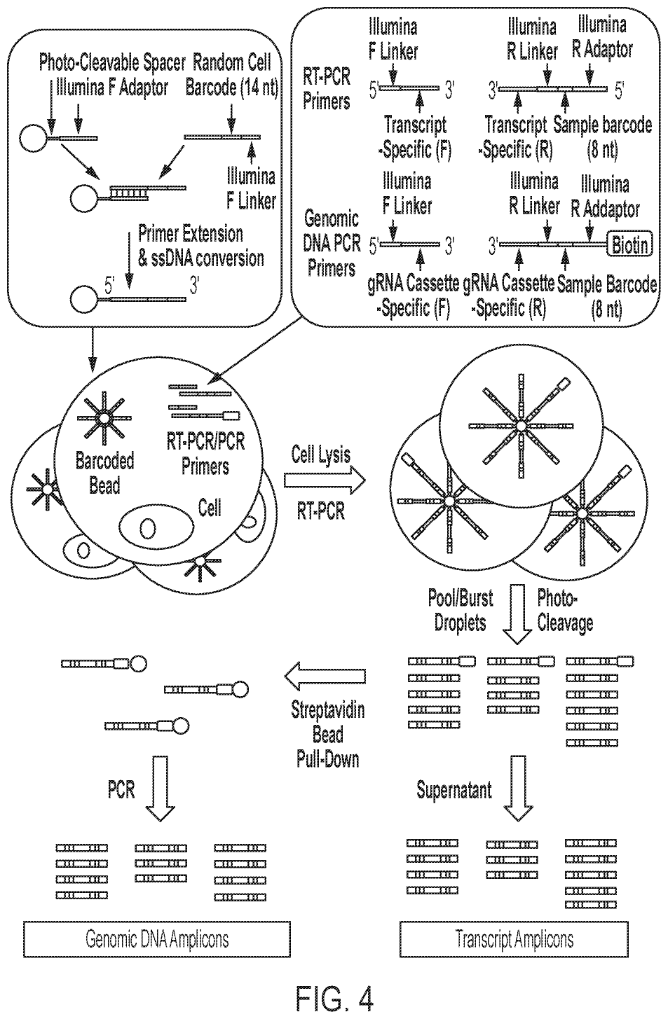

Also disclosed herein is a hybrid approach of combining targeted genomic DNA and mRNA amplicon sequencing, both the presence of multiple gRNAs/mutations and expression levels of selected transcript can be measured at a single-cell level as illustrated in FIG. 4, where biotinylated primers are used to separate DNA amplicons from mRNA amplicons. Furthermore, this platform can be readily scaled-up to accommodate extreme complexity. For example, the gut microbiome contains >10,000 detectable species, each with a few thousand genes, which makes it virtually impossible to profile the global gene expression levels and decompose the data to the species level for mechanistic studies. Conceptually, by targeted amplification of both genomic DNA (e.g., 16S rRNA gene) and cDNA (e.g., genes in a cancer drug-metabolizing pathway), species-specific gene expression profiles can be obtained that can be used for building a metabolic flux model by combining with metabolomics data. Further, immune receptor compositions, such as the specific pairing of alpha and beta subunit sequences of T cell receptors, in a cell population can be studies at a single cell level.

Disclosed herein is a method of determining functional genomics analysis on a population of cell. In embodiments, the method includes transducing a population of cells of interest with a set of nucleic acid molecules comprising a pooled library of genomic perturbagens having a mid-range multiplicity of infection (MOI) to create genome-integrated perturbagen cassettes, e.g., perturbagen cassettes that have been integrated into the genome of the cell population of interest. In embodiments, the cells with integrated perturbation cassettes are subjected to one or more rounds of phenotypical selection. In embodiments, the method includes separating each single cell from the population of cells individually into a set of compartments or droplets. Each of the compartments further includes a forward primer with a nucleic acid sequence that specifically binds a nucleic acid sequence on the nucleic acid molecules comprising a pooled library of genomic perturbagens and is capable of directing amplification of the nucleic acid molecules comprising a common or universal 5′ sequence of genomic perturbagen sequences and a compartment (droplet)-specific nucleic acid barcode that is unique to each compartment. Each compartment further includes a reverse primer with a nucleic acid sequence that specifically binds a nucleic acid sequence on the nucleic acid molecules comprising a common or universal 3′ sequence (opposite strand of the forward primer) of genomic perturbagen sequences and is capable of directing amplification of the nucleic acid molecules comprising the unique individual genomic perturbagen sequences. In embodiments, the method includes amplifying the genome-integrated perturbagen cassettes with the forward and reverse primers to create amplicons, wherein the amplicons comprise the nucleic acid sequence of the genome-integrated perturbagen cassette. In embodiments, the method further includes pooling the contents of the compartments and determining the sequence of the amplicons.

Transduction of cells at a higher multiplicity of infection (MOI) or delivering vectors by transfection at a higher MOI would result in any given cell receiving multiple perturbagens and allow the determination of the combinatorial effect of multiple perturbations. In embodiments, 2, or 3, or 4, or 5, or up to 10 genes, preferably 2-7 genes are perturbed in a single cell. In embodiments, the MOI is greater than about 0.5. In embodiments, the MOI is between about 1.0 and about 3.0. In embodiments, the pooled library of genomic perturbagens comprises a CRISPR guide RNA library (gRNA library). In embodiments, the pooled library of genomic perturbagens comprises an RNAi library, such as an shRNA library.

In embodiments, the method includes subjecting the population of cells of interest to one or more additional steps of mid-MOI transduction and phenotype selection.

In embodiments, the sequence of the amplicons are determined by nucleic acid sequencing, nucleic acid hybridization, or a combination thereof. In embodiments, the nucleic acid sequencing comprises pooled sequencing. Amplicons labeled nucleic acid barcodes can be formed and/or amplified by methods known in the art, such as polymerase chain reaction (PCR), for example the reverse and forward primers can be used for PCR amplification and subsequent high-throughput sequencing. In certain embodiments, the reverse and forward primers include or are linked to sequencing adapters (for example, universal primer recognition sequences) such that allow for amplification and sequencing (for example, P7, SBS3, and P5 elements for Illumina® sequencing).

The amplicons as described herein may be optionally sequenced by any method known in the art, for example, using methods of high-throughput sequencing, also known as next generation sequencing or deep sequencing. An genome-integrated perturbagen cassette labeled with a barcode can be sequenced with the barcode to produce a single read and/or contig containing the sequence, or portions thereof, of both the genome-integrated perturbagen cassette and the barcode. Exemplary next generation sequencing technologies include, for example, Illumina® sequencing, Ion Torrent sequencing, 454 sequencing, SOLiD sequencing, and nanopore sequencing amongst others.

In some embodiments, the sequence of barcode labeled genome-integrated perturbagen cassette is determined by non-sequencing based methods. For example, variable length probes or primers can be used to distinguish barcodes labeling distinct genome-integrated perturbagen cassette by, for example, the length of the barcodes, or the length of genome-integrated perturbagen cassette.

In some embodiments of the disclosed methods, determining the identity of a nucleic acid, such as a nucleic acid barcode or genome-integrated perturbagen cassette, includes detection by nucleic acid hybridization. Nucleic acid hybridization involves providing a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (for example, low temperature and/or high salt) hybrid duplexes (for example, DNA:DNA, RNA:RNA, or RNA:DNA) will form even where the annealed sequences are not perfectly complementary. Thus, specificity of hybridization is reduced at lower stringency. Conversely, at higher stringency (for example, higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNAse protection assays (Hod, Biotechniques 13 :852-4, 1992); and PCR-based methods, such as reverse transcription polymerase chain reaction (RT-PCR) (Weis et al., Trends in Genetics 8:263-4, 1992).

One of the superior properties of the disclosed methods is the samples, such as the contents of multiple compartments, can be analyzed together in a single reaction, for example a pooled reaction. Thus, in some examples, the individual compartments are pooled to create a pooled sample. The target molecules and/or target nucleic acids from a plurality of compartments, labeled according to the disclosed methods, can be combined to form a pool. For example, labeled target molecules and/or target nucleic acids in a plurality of emulsion droplets can be combined by breaking the emulsion. Thus, in some embodiments, the emulsion is broken. The pools can be comprised of labeled target molecules and/or target nucleic acids coming from a large number of individual compartments or discrete volumes (for example, at least 2, 3, 4, 5, 6, 7, 8, 9, 10, 20, 30, 40, 50, 100, 500, 1,000, 2,500, 5,000, 10,000, 50,000, 100,000, 500,000, 1,000,000, 2,000,000, or more; in various examples, for example, those utilizing plates, the numbers can be, for example, at least 6, 24, 96, 192, 384, 1,536, 3,456, or 9,600), thus facilitating processing of very large numbers of samples at the same time (for example, by highly multiplexed affinity measurement), leading to great efficiencies.

In embodiments, the compartments comprise droplets and the single cells of the population cells are encapsulated in the droplets. In embodiments, the droplets comprise an oil and water emulsion. In embodiments, the method includes coupling sequencing adapters to the application products.

In embodiments, the oligonucleotide forward primer is coupled to a solid substrate. In embodiments, the oligonucleotide forward primer is coupled to the solid substrate with a photo-cleavable DNA spacer. In embodiments, the photo-cleavable DNA spacer comprises acrydite-modified photo-cleavable DNA spacer. In embodiments, the solid substrate comprises a hydrogel bead.

In embodiments, the method is used in a functional screening study at a single cell level. In embodiments, the method is used at a single cell level to map which pathways are altered by mutations or gene expression, for example to determine tumor heterogeneity in aggressiveness and/or drug resistance. In embodiments, the method is used, for example, with T cell receptors, B cell receptors, TKRs, other cell receptors, etc., to determine which chains/subunits partner together in individual cells. Such analysis could have a major impact on tumor immunotherapy. In embodiments, the method is used to investigate clonal evolution of cancer cells, for example, by tracing mutational status of millions of cells. In embodiments, the method is used to study metabolic flux modeling of mammalian or bacterial cells at a single cell level, for example, when targeted DNA or RNA amplification of metabolic genes are combined with metabolomics and metagenomics measurements. In embodiments, the method is used for genome-wide screens to discover potential drug targets for cancer with specific set of mutations. In embodiments, the method is used for RNA sequencing to investigate expression profiles of a group of target genes, such as genes in a biological pathway of interest, at a single cell level in a heterogeneous population of cells, which can be done by adding a reverse transcription step prior to PCR with a set of gene-specific primers. In embodiments, as a hybrid approach of combining functional screening and RNA sequencing, the method is used to monitor expression changes of a targeted set of genes in a pooled perturbagen library-transduced population of cells at a single cell level. For example, from CRISPR gRNA library transduced cells, this method can identify a set of genes that affect in combination the activity of a biological pathway (e.g., p53 pathway) by reading the integrated gRNA sequences and measuring gene expression levels of a set of known genes (e.g., CDKN1A and BAX). In embodiments, this hybrid screen approach can be used to discover novel drug target genes that can activate or inactivate cellular pathways related to a broad range of human diseases, such as cancer, metabolic and neurodegenerative diseases.

In embodiments, the population of cells are derived from cell lines. In embodiments, the population of cells are primary cells, for example obtained from one or more subject or patients.

In embodiments, a method of functional genomics determination is disclosed including transducing a population of cells of interest with set of nucleic acid molecules, the set of nucleic acid molecules comprising a pooled library of genomic perturbagens having a mid-range multiplicity of infection (MOI) to create genome-integrated perturbagen cassettes; determining a phenotype of individual cells in the population of cells. The method also includes separating single cells of the population cells individually into a set of compartments, wherein each compartment includes: a genomic DNA forward primer with a nucleic acid sequence that specifically binds a nucleic acid sequence on the nucleic acid molecules comprising a common 5′ sequence of the genomic perturbagens, and a first linker nucleic acid sequence; and a genomic DNA reverse primer with a nucleic acid sequence that specifically binds a nucleic acid sequence on the nucleic acid molecules comprising a common 3′ sequence (opposite strand of the forward primer) of the genomic perturbagen sequences, a second linker nucleic acid sequence, a sample barcode nucleic acid sequence, and a sequencing adaptor associated with either the genomic DNA forward primer or reverse primer; and a compartment specific nucleic acid, comprising a compartment specific nucleic acid barcode that is unique to each compartment, a forward sequencing adaptor, and the first linker nucleic acid sequence or second linker nucleic acid sequence. For example, the sample barcode nucleic acid sequence and sequencing adapter are associated with the genomic DNA forward primer. the sample barcode nucleic acid sequence and sequencing adapter are associated with the genomic DNA reverse primer.

The method also includes: amplifying the genome-integrated perturbagen cassettes by RT-PCR with the genomic DNA forward primer and the genomic DNA reverse primer to create genomic perturbagen amplicons; pooling the contents of the compartments; and determining the sequence of the genomic perturbagen amplicons.

In some embodiments, the compartments of the disclosed method further include a RTC-PCR transcript specific primer pair. The RTC-PCR transcript specific primer pair can include a RTC-PCR forward primer with a nucleic acid sequence that specifically binds a 5′ transcript specific nucleic acid sequence and the first linker nucleic acid sequence; and a RTC-PCR reverse primer with a nucleic acid sequence that specifically binds a 3′ transcript specific nucleic acid sequence and the second linker nucleic acid sequence. In some embodiments, the sample barcode nucleic acid sequence, and/or sequencing adaptor specifically bind to the RTC-PCR forward primer. In some embodiments, the sample barcode nucleic acid sequence and/or sequencing adaptor specifically bind to RTC-PCR reverse primer. In some embodiments, the method further includes amplifying the mRNA by RT-PCR with the RTC-PCR forward primer and the RTC-PCR reverse primer to create transcript amplicons; and determining the sequence of the transcript amplicons.

In some embodiments, the genomic DNA reverse primer includes a capture moiety, such as biotin. For example, the method can further separating biotin labeled nucleic acids from non-biotin labeled nucleic acids. In some embodiments of this method, the MOI is greater than about 0.5, such as between about 1.0 and about 3.0. In some embodiments, the pooled library of genomic perturbagens includes a CRISPR guide RNA library (gRNA library). In some embodiments, the pooled library of genomic perturbagens includes an RNAi library, such as an shRNA library. In some embodiments, the pooled library of genomic perturbagens includes an gene-overexpressing library. In some embodiments, the method further includes subjecting the population of cells of interest to one or more additional steps of mid-MOI transduction and phenotype selection. In some embodiments, the sequence of the amplification products is determined by nucleic acid sequencing, nucleic acid hybridization or a combination thereof. For example, the nucleic acid sequencing includes pooled sequencing. In some examples, the method includes compartments including droplets, such as oil and water emulsion, and wherein the single cells of the population cells are encapsulated in the drops. In some examples, the method includes a compartment specific nucleic acid coupled to a solid substrate, such as with a photo-cleavable DNA spacer (e.g., a photo-cleavable DNA spacer including a acrydite-modified photo-cleavable DNA spacer). In some embodiments, the solid substrate includes a hydrogel bead. In some examples, the disclosed method is used in a functional screening study at a single cell level. For example, the population of cells are derived from cell lines or primary cells.

Various compositions and methods of use related to the delivery, engineering, optimization and therapeutic applications of systems, methods, and compositions used for the control of gene expression involving sequence targeting, such as genome perturbation or gene-editing, may be utilized in this disclosure. In certain embodiments, the perturbagens include a gene editing system, such as a CRISPR nuclease system, a meganuclease system, a zinc finger nuclease system (ZFN) or a transcription activator-like effector-based nuclease (TALEN) system.

Since 2013, the CRISPR nuclease system has been used for gene editing (adding, disrupting or changing the sequence of specific genes) and gene regulation in species throughout the tree of life. By delivering the Cas enzyme and appropriate guide RNAs into a cell, the organism's genome can be cut at any desired location. It may be possible to use CRISPR to build RNA-guided gene drives capable of altering the genomes of entire populations. Nuclease enzymes and CRISPR nuclease systems, including Cpf1 enzymes are known in the art, see US Patent Publication No. US20160208243 which is hereby incorporated herein by reference in its entirety. “CRISPRs (clustered regularly interspaced short palindromic repeats)” are DNA loci containing short repetitions of base sequences. Each repetition is followed by short segments of “spacer DNA” from previous exposures to a virus. CRISPRs are found in approximately 40% of sequenced bacteria genomes and 90% of sequenced archaea. CRISPRs are often associated with cas genes that code for proteins related to CRISPRs. The CRISPR nuclease system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms.

In one aspect, the genome perturbation or gene-editing relates to CRISPR and components thereof. The CRISPR-Cas system does not require the generation of customized proteins to target specific sequences, but rather a single Cas enzyme can be programmed by a short guide RNA molecule to recognize a specific DNA target. The CRISPR-Cas systems of bacterial and archaeal adaptive immunity show extreme diversity of protein composition and genomic loci architecture. The CRISPR-Cas system loci has more than 50 gene families and there is no strictly universal genes indicating fast evolution and extreme diversity of loci architecture. So far, adopting a multi-pronged approach, there is comprehensive cas gene identification of about 395 profiles for 93 Cas proteins. Classification includes signature gene profiles plus signatures of locus architecture. A new classification of CRISPR-Cas systems is proposed in which these systems are broadly divided into two classes, Class 1 with multi-subunit effector complexes and Class 2 with single-subunit effector modules exemplified by the Cas9 protein. Novel effector proteins associated with Class 2 CRISPR-Cas systems may be developed as powerful genome engineering tools and the prediction of putative novel effector proteins and their engineering and optimization is important. In addition to the Class 1 and Class 2 CRISPR-Cas systems, more recently a putative Class 2, Type V CRISPR-Cas effector proteins have been discovered as exemplified by Cpf1. Examples of useful CRISPR-Cas systems and components include, but are not limited to, the components, or any corresponding orthologs thereof, and delivery of such components, including methods, materials, delivery vehicles, vectors, particles, and making and using thereof, as described in, e.g., U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 Al (U.S. application Ser. No. 14/213,991), US 2014-0273234 Al (U.S. application Ser. No. 14/293,674), US2014-0273232 Al (U.S. application Ser. No. 14/290,575), US 2014-0273231 (U.S. application Ser. No. 14/259,420), US 2014-0256046 Al (U.S. application Ser. No. 14/226,274), US 2014-0248702 Al (U.S. application Ser. No. 14/258,458), US 2014-0242700 Al (U.S. application Ser. No. 14/222,930), US 2014-0242699 Al (U.S. application Ser. No. 14/183,512), US 2014-0242664 Al (U.S. application Ser. No. 14/104,990), US 2014-0234972 Al (U.S. application Ser. No. 14/183,471), US 2014-0227787 Al (U.S. application Ser. No. 14/256,912), US 2014-0189896 Al (U.S. application Ser. No. 14/105,035), US 2014-0186958 (U.S. application Ser. No. 14/105,017), US 2014-0186919 Al (U.S. application Ser. No. 14/104,977), US 2014-0186843 Al (U.S. application Ser. No. 14/104,900), US 2014-0179770 Al (U.S. application Ser. No. 14/104,837) and US 2014-0179006 Al (U.S. application Ser. No. 14/183,486), US 2014-0170753 (U.S. application Ser. No. 14/183,429); European Patents EP 2 784 162 Bl and EP 2 771 468 Bl; European Patent Applications EP 2 771 468 (EP13818570.7), EP 2 764 103 (EP 13824232.6), and EP 2 784 162 (EP14170383.5), and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809) , PCT/US2014/62558. Each of the aformentioned patents, patent publications, and applications are incorporated by reference herein by reference in their entireties.

Also with respect to general information on CRISPR-Cas Systems, mention is made of the following (also hereby incorporated herein by reference): Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science Febuary 15; 339(6121):819-23 (2013); RNA-guided editing of bacterial genomes using CRISPR-Cas systems. Jiang W., Bikard D., Cox D., Zhang F, Marraffini LA. Nat Biotechnol Mar; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states Konermann S, Brigham M D, Trevino A E, Hsu P D, Heidenreich M, Cong L, Piatt R J, Scott D A, Church G M, Zhang F. Nature. August 22; 500(7463):472-6. doi: 10.1038/Naturel2466. Epub 2013 Aug. 23 (2013); Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013); Genome engineering using the CRISPR-Cas9 system. Ran, F A., Hsu, P D., Wright, J., Agarwala, V., Scott, D A., Zhang, F. Nature Protocols November; 8(11):2281-308 (2013-B) Genome-Scale CRISPR-Cas9 Knockout Screening in Human Cells. Shalem, O., Sanjana, N E., Harteman, E., Shi, X., Scott, D A., Mikkelson, T., Heckl, D., Ebert, B L., Root, D E., Doench, J G., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., lshitani, R., Zhang, F., Nureki, O. Cell Febuary 27, 156(5):935-49 (2014); Genome-wide binding of the CRISPR endonuclease Cas9 in mammalian cells. Wu X., Scott D A., Kriz A J., Chiu A C, Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014); CRISPR-Cas9 Knockm Mice for Genome Editing and Cancer Modeling. Piatt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp PA, Zhang F. Cell 159(2): 440-455 DOI: 10.1016/j.cell.2014.09.014(2014); Development and Applications of CRISPR-Cas9 for Genome Engineering, Hsu P D, Lander E S, Zhang F., Cell. June 5; 157(6): 1262-78 (2014); Genetic screens in human cells using the CRISPR/Cas9 system, Wang T, Wei J J, Sabatini D M, Lander E S., Science. January 3; 343(6166): 80-84. doi: 10.1126/science.1246981 (2014); Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12): 1262-7 (2014); In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(I): 102-6 (2015); Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015); A split-Cas9 architecture for inducible genome editing and transcription modulation, Zetsche B, Volz SE, Zhang F., (published online 2 Feb. 2015) Nat Biotechnol. Febuary; 33(2): 139-42 (2015); Genome-wide CRISPR Screen in a Mouse Model of Tumor Growth and Metastasis, Chen S, Sanjana NE, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F, (published online 1 Apr. 2015), Nature. April 9; 520(7546): 186-91 (2015) each of which is incorporated herein by reference in its entirety.

The compartments, such as discrete volumes or spaces, as disclosed herein mean any sort of area or volume which can be defined as one where a cell of interest, or forward and reverse nucleic acid primers are not free to escape or move between. Compartments include droplets, such as the droplets from a water-in-oil emulsion, or as deposited on a surface, such as a microfluidic droplet, for example deposited on a slide. Other types of compartments include without limitation a tube, well, plate, pipette, pipette tip, and bottle. Other types of compartments include “virtual” containers, such as defined by areas exposed to light, diffusion limits, or electro-magnetic means. Such compartments can also exist by diffusion defined volumes, or spaces that are only accessible to certain molecules or reactions because diffusion constraints effectively defining a space, for example, chemically defined volumes or spaces where only certain target molecules can exist because of their chemical or molecular properties such as size, or electro-magnetically defined volumes or spaces where the electro-magnetic properties of the target molecules or their supports such as charge or magnetic properties can be used to define certain regions in a space. Such discrete may also be optically defined volumes or spaces that may be defined by illuminating it with visible, ultraviolet, infrared, or other wavelengths of light such that only target molecules within the defined space may be labeled. Such compartments can be composed of, for example, plastic, metal, composite materials, and/or glass. Such compartments can be adapted for placement into a centrifuge (for example, a microcentrifuge, an ultracentrifuge, a benchtop centrifuge, a refrigerated centrifuge, or a clinical centrifuge). A discreet volume can exist on its own, as a separate entity, or be part of an array of such discreet volumes, for example, in the form of a strip, a microwell plate, or a microtiter plate. A compartment can have a capacity of, for example, at least about 1 femtoliter (fl) to about 1000 ml, such as about 1 fl, 10 fl, 100 fl, 250 fl, 500 fl, 750 fl, 1 picoliter (pi), 10 pi, 100 pi, 250 pi, 500 pi, 750 pi, 1 nl, 10 nl, 100 nl, 250 nl, 500 nl, 750 nl, 1 μl, 5 μl, 10 μl, 20 μl, 25 μl, 50 μl, 100 μl, 200 μl, 250 μl, 500 μl, 750 μl, 1 ml, 1.25 ml, 1.5 ml, 2 ml, 2.5 ml, 5 ml, 10 ml, 15 ml, 20 ml, 25 ml, 50 ml, 100 ml, 150 ml, 200 ml, 250 ml, 300 ml, 350 ml, 400 ml, 450 ml, 500 ml, 550 ml, 600 ml, 650 ml, 700 ml, 750 ml, 800 ml, 900 ml, or 1000 ml.

In certain embodiments, a compartment is a droplet, such as a droplet in an emulsion and/or a microfluidic droplet. Emulsification can be used in the methods of the disclosure to separate or segregate a sample or set of samples into a series of compartments, for example a compartment having a single cell. Typically, as used in conjunction with the methods and compositions disclosed herein, an emulsion will include a plurality of droplets, each droplet including a single cells and a forward primer including a nucleic acid barcode, such that each droplet includes a unique barcode that distinguishes it from the other droplets. Droplets in an emulsion can be sorted and/or isolated according to methods well known in the art. For example, double emulsion droplets containing a fluorescence signal can be analyzed and/or sorted using conventional fluorescence-activated cell sorting (FACS) machines at rates of >10⁴ droplets. However, the emulsions are highly polydisperse, limiting quantitative analysis, and it is difficult to add new reagents to pre-formed droplets (Griffiths et al., Trends Biotechnol 24(9):395-402, 2006). These limitations can, however, be overcome by using protocols based on droplet-based microfluidic systems (see for example Teh et al., Lab on a chip 8(2): 198-220, 2008; Theberge et al., Angew Chem Int Ed Engl 49(34):5846-5868, 2010; and Guo et al., Lab on a chip 12(12):2146, 2012) in which highly monodisperse droplets of picoliter volume can be made (Anna et al., Appl Phys Lett 82(3):364-366, 2003), fused (Song et al., Angew Chem Int Edit 42(7):767-772, 2003; Chabert et al., Electrophoresis 26(19):3706-3715, 2005), split (Song et al., Angew Chem Int Edit 42(7):767-772, 2003; Link et al., Phys Rev Lett 92(5):054503, 2004), incubated (Song et al., Angew Chem Int Edit 42(7):767-772, 2003; Frenz et al., Lab on a chip 9(10): 1344-1348, 2009), and sorted triggered on fluorescence (Beret, et al, Lab on a chip 9(13): 1850-1858, 2009), at kHz frequencies, such as those described in Mazutis et al. (Nat. Protoc. 8(5): 870-891, 2013), incorporated by reference herein. As disclosed herein, an emulsion can include various compounds, enzymes, or reagents in addition to single cells and primers. These additives may be included in the emulsion solution prior to emulsification. Alternatively, the additives may be added to individual droplets after emulsification.

Emulsion may be achieved by a variety of methods known in the art (see, for example, US 2006/0078888 Al, of which paragraphs [0139]-[0143] are incorporated by reference herein). In some embodiments, the emulsion is stable to a denaturing temperature, for example, to 95° C. or higher. An exemplary emulsion is a water-in-oil emulsion. In some embodiments, the continuous phase of the emulsion includes a fluorinated oil. An emulsion can contain a surfactant or emulsifier (for example, a detergent, anionic surfactant, cationic surfactant, or amphoteric surfactant) to stabilize the emulsion. Other oil/surfactant mixtures, for example, silicone oils, may also be utilized in particular embodiments. An emulsion can be contained in a well or a plurality of wells, such as a plate, for easy of handling. In some examples, one or more target molecules, target nucleic acid and nucleic acid barcodes are compartmentalized. An emulsion can be a monodisperse emulsion or a polydisperse emulsion.

Compartmentalization of target molecules, target nucleic acids and nucleic acid barcodes into wells can be achieved, in some embodiments, due to physical limitations relating to the mass or dimensions of the target molecules and nucleic acid barcodes, the dimensions of the well, or a combination thereof. A well may be a fiberoptic faceplate where the central core is etched with an acid, such as an acid to which the core-cladding is resistant. A well may be a molded well. The wells may be covered to prevent communication between the wells, such that the beads present in a particular well remain within the well or are inhibited from moving into a different well. The cover may be a solid sheet or physical barrier, such as a neoprene gasket, or a liquid barrier, such as fluorinated oil. Methods applicable to the present disclosure are known in the art (for example, Shukla et al., J. Drug Targeting 13 : 7-18, 2005; Koster et al., Lab on a Chip 8: 1110-1115, 2008).

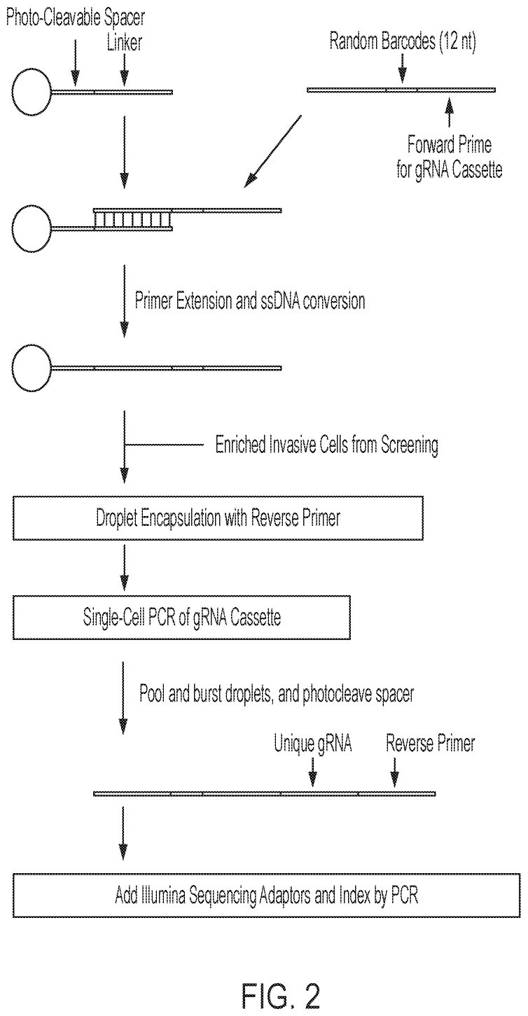

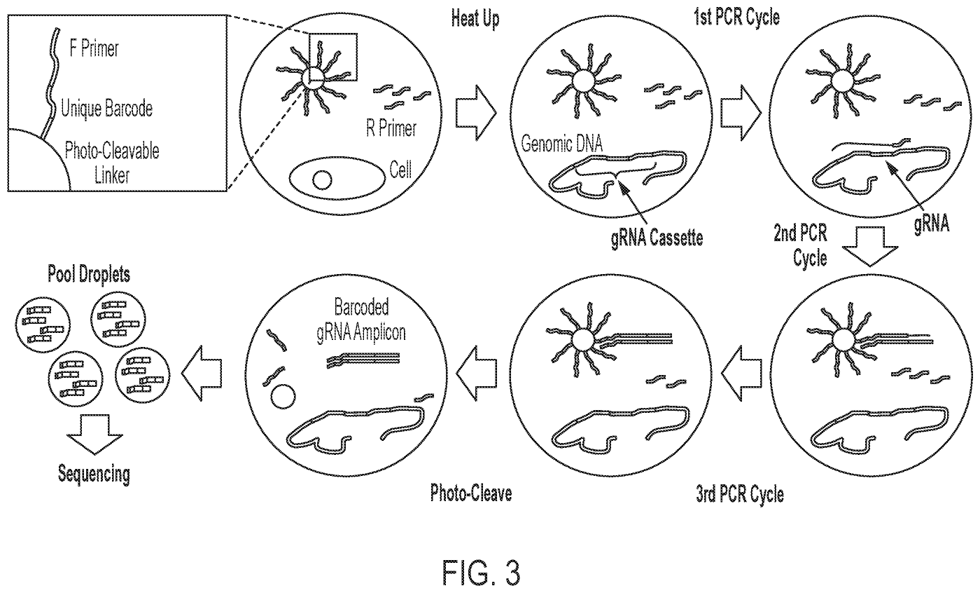

In certain embodiments, the single cells or a portion of the acellular system from the sample are encapsulated together with a bead, such as a hydrogel bead that includes the forward primer with a nucleic acid barcode reversibly coupled thereto. A set of hydrogel beads, such as PEG-DA beads, of uniform size is created, for example, using a PDMS chip. In some embodiments, the uniformly sized PEG-DA hydrogel bead are co-polymerized with a generic capture oligonucleotide, which can be used to build a nucleic acid identification sequence unique to each bead. Using automation techniques and split-pool labeling (see, for example, International Patent Publication No. WO2014/047561, which is specifically incorporated by reference) a unique nucleic acid barcode can be added to each bead. Using microfluidics, the individual beads can be placed into single drop and then single cells added, such that each drop in the emulsion contains a single cell and single hydrogel bead containing a unique nucleic acid bar code. As shown in the FIG. 3, this system can be used to label all of the amplicons derived from a cell with a unique barcode. If the emulsion is then broken, the result is a pooled sample of amplicons barcoded according to droplet. Thus, all of the amplicons can be traced back to the single cell from which they originated. As exemplified in FIG. 2, in some embodiments, a bead includes an exemplary bead and barcode for labeling an amplicon. In specific embodiments, the barcodes are delivered to the compartments by delivering a single bead to each compartment wherein each bead carries multiple copies of a single origin-specific barcode sequence.

In some embodiments of the method, the cells are contacted with one or more test agents, such as a small molecule, a nucleic acid, a polypeptide, or a polysaccharide.

Examples of test agents include small molecule compounds, nucleic acids, polypeptides (such as proteins, antibodies, antigens, and/or immunogens), or a polysaccharide. In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

The compounds identified using the methods disclosed herein can serve as conventional “lead compounds” or can themselves be used as potential or actual therapeutics. In some instances, pools of candidate agents can be identified and further screened to determine which individual or subpools of agents in the collective have a desired activity.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 108 samples to be screened in a single day.

Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

The manipulation of fluids to form fluid streams of desired configuration, discontinuous fluid streams, droplets, particles, dispersions, etc., for purposes of fluid delivery, product manufacture, analysis, and the like, is a relatively well-studied art. Microfluidic systems have been described in a variety of contexts, typically in the context of miniaturized laboratory (e.g., clinical) analysis. Other uses have been described as well. For example, WO 2001/89788; WO 2006/040551; U.S. Patent Application Publication No. 2009/0005254; WO 2006/040554; U.S. Patent Application Publication No. 2007/0184489; WO 2004/002627; U.S. Pat. No. 7,708,949; WO 2008/063227; U.S. Patent Application Publication No. 2008/0003142; WO 2004/091763; U.S. Patent Application Publication No. 2006/0163385; WO 2005/021151; U.S. Patent Application Publication No. 2007/0003442; WO 2006/096571; U.S. Patent Application Publication No. 2009/0131543; WO 2007/089541; U.S. Patent Application Publication No. 2007/0195127; WO 2007/081385; U.S. Patent Application Publication No. 2010/0137163; WO 2007/133710; U.S. Patent Application Publication No. 2008/0014589; U.S. Patent Application Publication No. 2014/0256595; and WO 2011/079176. In a preferred embodiment, single cell analysis is performed in droplets using methods according to WO 2014085802. Each of these aforementioned patents and publications is herein incorporated by reference in its entirety.

Single cells may be sorted into separate compartments, such as droplets, by dilution of the sample and physical movement, such as pipetting. A machine can control the pipetting and separation. The machine may be a computer controlled robot.

Microfluidics may also be used to separate the single cells. Single cells can be separated using microfluidic devices. Microfluidics involves micro-scale devices that handle small volumes of fluids. Because microfluidics may accurately and reproducibly control and dispense small fluid volumes, in particular volumes less than 1 pl, application of microfluidics provides significant cost-savings. The use of microfluidics technology reduces cycle times, shortens time-to-results, and increases throughput. The small volume of microfluidics technology improves amplification and construction of DNA libraries made from single cells. Furthermore, incorporation of microfluidics technology enhances system integration and automation.

Single cells may be divided into single droplets using a microfluidic device. The nucleic acid from the single cells in such droplets may be further labeled with a nucleic acid barcode. In this regard reference is made to Macosko et al., 2015, “Highly Parallel Genome-wide Expression Profiling of Individual Cells Using Nanoliter Droplets” Cell 161, 1202-1214 and Klein et al., 2015, “Droplet Barcoding for Single-Cell Transcriptomics Applied to Embryonic Stem Cells” Cell 161, 1187-120,1 all the contents and disclosure of each of which are herein incorporated by reference in their entirety. Not being bound by a theory, the volume size of an aliquot within a droplet may be as small as 1 fl.

Single cells may be diluted into a physical multi-well plate or a plate free environment. The multi-well assay modules (e.g., plates) may have any number of wells and/or chambers of any size or shape, arranged in any pattern or configuration, and be composed of a variety of different materials. Multi-well assay plates that use industry standard multi-well plate formats for the number, size, shape and configuration of the plate and wells are preferred. Examples of standard formats include 96-, 384-, 1536- and 9600-well plates, with the wells configured in two-dimensional arrays. Other formats include single well, two well, six well and twenty-four well and 6144 well plates.

In embodiments, for more high throughput processing, one or more microfluidic chips can be used to capture the cells in nanoliter-sized aqueous droplets (Macosko et al., 2015, “Highly Parallel Genome-wide Expression Profiling of Individual Cells Using Nanoliter Droplets” Cell 161, 1202-1214). The aqueous droplets or microwells may be simultaneously loaded with barcoded beads, each of which has oligonucleotides including; a “cell barcode” that is the same across all the primers on the surface of any one bead, but different from the cell barcodes on all other beads; a Unique Molecular Identifier (UMI), different on each primer, that enables sequence reads derived from the same original DNA tag (amplification and PCR duplicates) to be identified computationally. Once the beads are loaded, they can be pooled for amplification and library preparation, and sequencing.

In another aspect, the present invention provides screening methods to determine the effect on protein, post translational modifications and cellular constituents of single cells or isolated aggregations of cellular constituents in response to the perturbation of genes or cellular circuits. Perturbation may be knocking down a gene, increasing expression of a gene, mutating a gene, mutating a regulatory sequence, or deleting non-protein-coding DNA.

In one embodiment, CRISPR/Cas9 may be used to perturb protein-coding genes or non-protein-coding DNA. CRISPR/Cas9 may be used to knockout protein-coding genes by frameshifts, point mutations, inserts, deletions, or to induce gene expression by using modified Cas9 proteins. An extensive toolbox may be used for efficient and specific CRISPR/Cas9 mediated knockout as described herein, including a double-nicking CRISPR to efficiently modify both alleles of a target gene or multiple target loci and a smaller Cas9 protein for delivery on smaller vectors (Ran, F. A. , et al., In vivo genome editing using Staphylococcus aureus Cas9. Nature. 520, 186-191 (2015)).

In one embodiment, perturbation of genes is by RNAi. The RNAi may be shRNA's targeting genes. The shRNAs may be delivered by any methods known in the art. In one embodiment the shRNAs may be delivered by a viral vector. The viral vector may be a lentivirus.

In one embodiment, perturbation of genes is by overexpression. The gene-overexpressing perturbagens may be delivered by any methods known in the art. In one embodiment, the gene-overexpressing perturbagens may be delivered by a viral vector. The viral vector may be a lentivirus.

In one embodiment, a CRISPR based pooled screen is used. Perturbation may rely on gRNA expression cassettes that are stably integrated into the genome. The expressed gRNA may serve as a molecular barcode, reporting the loss of function of the target in a cell. Alternatively, optimized separate barcodes may be co-expressed with the gRNA.

This disclosure is primarily designed for genome-wide screen to discover potential drug targets for cancer with specific set of mutations, which can be developed as a service for the Genomics/Bioinformatics Cores, as an example. Although cell lines are being mostly targeted, this method can also be applied to patient-derived cells to screen and identify genes that can be targeted by drugs. Alternatively, based on the mutation profile (e.g., point mutations, amplifications, and deletions) of a given patient, a cell line with a core set of driver mutations (i.e., major oncogenes and tumor suppressor mutations) can be engineered by CRISPR-based gene editing and/or lentiviral overexpression technologies, and screened for drug targetable co-drivers to guide the selection of drug-targetable pathways and genes.

### Kits

The disclosure also provides kits containing any one or more of the elements disclosed in the methods and compositions herein. Elements may be provided individually or in combinations, and may be provided in any suitable container, such as a vial, a bottle, a bag or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers.

The following examples are provided to illustrate certain particular features and/or embodiments. These examples should not be construed to limit the disclosure to the particular features or embodiments described.

### EXAMPLES

### Example 1

Currently available single cell sequencing technologies, such as Chromium (10X Genomics), C1 (Fluidigm), Drop-Seq (Macosko, E. Z., et al., Highly Parallel Genome-wide Expression Profiling of Individual Cells Using Nanoliter Droplets. Cell, 2015. 161(5): p. 1202-14), and inDrop (Klein, A. M., et al., Droplet barcoding for single-cell transcriptomics applied to embryonic stem cells. Cell, 2015. 161(5): p. 1187-201), Zilionis, R., et al., Single-cell barcoding and sequencing using droplet microfluidics. Nat Protoc, 2017. 12(1): p. 44-73), are targeted to only read genome-scale information such as a whole genome or a whole transcriptome from up to thousands of cells, or selected DNA sequences in tumor samples for clinical purposes. Therefore, for screening applications that require testing millions of perturbagens at once, the current single cell sequencing platforms are severely under-powered.

Because typical perturbagens such as shRNA, cDNA, or gRNA share common sequences, such as linkers and antibiotics resistance genes, along with unique sequences, the genome-integrated perturbagen cassettes will be amplified by PCR based on universal primer sets, and only the short amplicons (not the entire genome) sequenced by pooled sequencing. To uniquely label each cell, single cells will be encapsulated within droplets by a microfluidic device. Cell-specific random barcode sequences will be added to the amplicons during the PCR step. The droplets will then be pooled and sequenced. Considering the capacity of currently available sequencers (e.g., 400 million read output for Illumina NextSeq), millions of perturbagens can be tested and quantified with enough sequencing depths (typically >100X) to provide adequate statistical power.

Based on previous literature (Macosko, E. Z., et al., Highly Parallel Genome-wide Expression Profiling of Individual Cells Using Nanoliter Droplets. Cell, 2015. 161(5): p. 1202-14, Zilionis, R., et al., Single-cell barcoding and sequencing using droplet microfluidics. Nat Protoc, 2017. 12(1): p. 44-731, a single droplet generator can generate approximately 10,000 usable (i.e., with a cell and a bead) droplets per hour. Since a combinatorial screen of genome-wide perturbagen library (i.e., one or more for each of 20,000 genes) is performed in a single cell, the diversity of combination can be very high. For example, if there are a total of 1,000 perturbagens that can exert functional effects in combinations, the diversity can reach 10⁶ and 10⁹ in case of 2-gene or 3-gene combinations, respectively. Therefore, to achieve enough coverage and identify a large number of co-existing perturbagen combinations in each cell by Amp-Drop-Seq, a multiplexed platform was developed with a throughput producing at least 4×10⁵ droplets within 2 hours. In specifics, up to 20 individual microfluidic devices will be multiplexed on a single platform. For screening, each pool of cells will be aliquoted and undergo 3 successive rounds of droplet encapsulation to obtain a total of >1 million droplets.

Hydrogel beads with sequencing adaptors and random barcodes for identification of individual cells will be generated based on the designs by Macosko et al (Macosko, E. Z., et al., Highly Parallel Genome-wide Expression Profiling of Individual Cells Using Nanoliter Droplets. Cell, 2015. 161(5): p. 1202-14), Zilionis et al (Zilionis, R., et al., Single-cell barcoding and sequencing using droplet microfluidics. Nat Protoc, 2017. 12(1): p. 44-73), and Klein et al (Klein, A. M., et al., Droplet barcoding for single-cell transcriptomics applied to embryonic stem cells. Cell, 2015. 161(5): p. 1187-201) with modifications, including an addition of the forward primer for perturbagen cassette amplification. Hydrogel beads will be used that can accommodate more DNA attachment sites (>10⁹) (Zilionis, R., et al., Single-cell barcoding and sequencing using droplet microfluidics. Nat Protoc, 2017. 12(1): p. 44-73) than solid beads, and thus provide robust amplification. The beads with 70 μm in diameter will be generated with a microfluidics device with acrydite-modified photo-cleavable DNA spacer, and the oligo pool with random barcodes for cell identification (12 nt, to be obtained from commercial sources such as IDT) will be added by primer extension (FIG. 2), which will have a diversity of 4¹² or ˜1.6×10⁷.

For transduction of pooled library of perturbagens such as gRNA libraries, instead of a low MOI of 0.3 to ensure that only one gRNA is integrated into a cell, a disclosed ‘shotgun screen’ utilizes mid to high MOI that allow transduction of multiple gRNAs in to a cell. To maximize the fraction of cells transduced with 1 or 2 gRNAs, a MOI of 2.0 will be used, where 31% each of total transduced cells will receive 1 and 2 gRNAs. When 400 million cells are transduced (i.e., 31% or 125 million cells are transduced with 2 gRNA), 2 gRNA combinations of 10,000 and 16,000 genes would be represented with approximately 2.5× and 1.0× coverage, respectively. To introduce more gRNAs, after the screen (e.g., selection of invasive cells), the entire pool of invasive cells (i.e., without clonal selection) can be subjected to another round of mid-MOI transduction and selection. As the result, 10%, 20%, and 23% of transduced cells in the final pool are expected to have 2, 3, or 4 gRNAs, respectively.

After the phenotypic screen, cells are subjected to droplet encapsulation, where one cell and one barcoded beads go into a droplet (FIG. 3). After PCR reaction to amplify the perturbagen cassettes and attach the barcodes, the amplicons are released from the beads by photo-cleavage. Then, the droplets are burst and pooled for sequencing. The Illumina sequencing adapters can be added either in the droplet or to the pooled amplicons. By adding different Illumina index sequences during the sequencing adaptor ligation step, samples from multiple experiments can be multiplexed.

The screening protocol is modified to read simultaneously the gRNA cassettes from genomic DNA and the levels of mRNA of selected genes (e.g. genes in a pathway of interest). Since reverse transcriptase is heat sensitive, mild detergents (e.g. IGEPAL) and mild heating (up to 50° C.) can be used for optimal release of genomic DNA and mRNA. Reverse transcription is then performed at 48° C. for 30 mins, followed by PCR to simultaneously amplify and barcode single-stranded cDNA and gRNA cassettes from genomic DNA (FIG. 4). Due to the imbalance between cDNA and gDNA amplicons (e.g., 1,000 copies×4 mRNAs vs. 2 copies×4 genome-integrated gRNA cassettes=4,000:8 ratio), it would be unlikely to identify all gRNAs coexisting in a single cell when the pooled droplets are sequenced at a depth of ˜1,000 reads per droplet (assuming 10⁹ reads for a pool of 10⁶ droplets). Therefore, 5′-biotinylated reverse primers for genomic DNA PCR (FIG. 4) can be used to separate and selectively amplify genomic DNA-derived from the mRNA-derived amplicons by avidin-coated beads, allowing detection of gRNA amplicon sequences, from which gRNA and mRNA amplicons will be mapped to a single cell via a common droplet barcode.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method of functional genomics determination, comprising:
transducing a population of cells of interest with set of nucleic acid molecules, the set of nucleic acid molecules comprising a pooled library of genomic perturbagens having a mid-range multiplicity of infection (MOI) to create genome-integrated perturbagen cassettes;
determining a phenotype of individual cells in the population of cells;
separating single cells of the population cells individually into a set of compartments, wherein each compartment further comprises:
a nucleic acid oligonucleotide, comprising:
a forward primer with a nucleic acid sequence that specifically binds a nucleic acid sequence on the nucleic acid molecules comprising a common 5′ sequence of the genomic perturbagens and a nucleic acid barcode; and
a compartment specific nucleic acid barcode that is unique to each compartment; and
a reverse primer with a nucleic acid sequence that specifically binds a nucleic acid sequence on the nucleic acid molecules comprising a common 3′ sequence (opposite strand of the forward primer) of the genomic perturbagen sequences;
amplifying the genome-integrated perturbagen cassettes with the forward primer and the reverse primer to create amplicons, wherein the amplicons comprise the nucleic acid sequence of the genome-integrated perturbagen cassette;
pooling the contents of the compartments; and
determining the sequence of the amplicons.

2. The method of claim 1, wherein the MOI is greater than about 0.5.

3. The method of claim 1, wherein the MOI is between about 1.0 and about 3.0.

4. The method of claim 1, wherein the pooled library of genomic perturbagens comprises a CRISPR guide RNA library (gRNA library), an RNAi library, such as an shRNA library and/or a gene-overexpressing library.

5. (canceled)

6. (canceled)

7. The method of claim 1, further comprising subjecting the population of cells of interest to one or more additional steps of mid-MOI transduction and phenotype selection.

8. The method of claim 1, wherein the sequence of the amplification products is determined by nucleic acid sequencing, nucleic acid hybridization or a combination thereof.

9. The method of claim 8, wherein the nucleic acid sequencing comprises pooled sequencing.

10. The method of claim 1, wherein the compartments comprise droplets and wherein the single cells of the population cells are encapsulated in the drops.

11. The method of claim 10, wherein the droplets comprise an oil and water emulsion.

12. The method of claim 1, further comprising coupling sequencing adapters to the amplicons.

13. The method of claim 1, wherein the forward primer is coupled to a solid substrate, such as with a photo-cleavable DNA spacer.

14. (canceled)

15. (canceled)

16. The method of claim 13, wherein the solid substrate comprises a hydrogel bead.

17. The method of claim 1, wherein the method is used in (1) a functional screening study at a single cell level; (2) at a single cell level, mapping which pathways are altered by mutations or gene expression, for example, to determine tumor heterogeneity in aggressiveness and drug resistance cancer; (3) determining which chains/subunits partner together in individual cells; (4) investigating clonal evolution of cancer cells by tracing mutational status of millions of cells; (5) studying a metabolic flux modeling of mammalian or bacterial cells at a single cell level; and/or (6) screening a genome to identify potential drug targets for cancer.

18. (canceled)

19. (canceled)

20. (canceled)

21. (canceled)

22. (canceled)

23. The method of claim 1, wherein the population of cells are derived from cell lines.

24. The method of claim 1, wherein the population of cells are primary cells.

25. A method of functional genomics determination, comprising:
transducing a population of cells of interest with set of nucleic acid molecules, the set of nucleic acid molecules comprising a pooled library of genomic perturbagens having a mid-range multiplicity of infection (MOI) to create genome-integrated perturbagen cassettes;
determining a phenotype of individual cells in the population of cells;
separating single cells of the population cells individually into a set of compartments, wherein each compartment comprises:
a genomic DNA forward primer with a nucleic acid sequence that specifically binds a nucleic acid sequence on the nucleic acid molecules comprising a common 5′ sequence of the genomic perturbagens, and a first linker nucleic acid sequence; and
a genomic DNA reverse primer with a nucleic acid sequence that specifically binds a nucleic acid sequence on the nucleic acid molecules comprising a common 3′ sequence (opposite strand of the forward primer) of the genomic perturbagen sequences, a second linker nucleic acid sequence,
a sample barcode nucleic acid sequence, and a sequencing adaptor associated with either the genomic DNA forward primer or reverse primer; and
a compartment specific nucleic acid, comprising a compartment specific nucleic acid barcode that is unique to each compartment, a forward sequencing adaptor, and the first linker nucleic acid sequence or second linker nucleic acid sequence;
amplifying the genome-integrated perturbagen cassettes by RT-PCR with the genomic DNA forward primer and the genomic DNA reverse primer to create genomic perturbagen amplicons; and
pooling the contents of the compartments;
determining the sequence of the genomic perturbagen amplicons.

26. The method of claim 25, wherein the compartments further comprise:
a RTC-PCR transcript specific primer pair, comprising:
a RTC-PCR forward primer with a nucleic acid sequence that specifically binds a 5′ transcript specific nucleic acid sequence and the first linker nucleic acid sequence; and
a RTC-PCR reverse primer with a nucleic acid sequence that specifically binds a 3′ transcript specific nucleic acid sequence and the second linker nucleic acid sequence,
wherein the sample barcode nucleic acid sequence, and sequencing adaptor specifically binds to either the RTC-PCR forward primer or RTC-PCR reverse primer;
the method further comprising amplifying the mRNA by RT-PCR with the RTC-PCR forward primer and the RTC-PCR reverse primer to create transcript amplicons; and
determining the sequence of the transcript amplicons.

27. The method of claim 25, wherein the genomic DNA reverse primer comprises a capture moiety, such as biotin.

28. (canceled)

29. The method of claim 27, further comprising separating biotin labeled nucleic acids from non-biotin labeled nucleic acids.

30. The method of claim 25, wherein the MOI is greater than about 0.5, such as between about 1.0 and about 3.0.

31. (canceled)

32. The method of claim 25, wherein the pooled library of genomic perturbagens comprises (1) a CRISPR guide RNA library (gRNA library); an RNAi library, such as an shRNA library; a gene-overexpressing library.

33. (canceled)

34. (canceled)

35. The method of claim 25, further comprising subjecting the population of cells of interest to one or more additional steps of mid-MOI transduction and phenotype selection.

36. The method of claim 25, wherein the sequence of the amplification products is determined by nucleic acid sequencing, nucleic acid hybridization or a combination thereof.

37. The method of claim 36, wherein the nucleic acid sequencing comprises pooled sequencing.

38. The method of claim 25, wherein the compartments comprise droplets and wherein the single cells of the population cells are encapsulated in the drops.

39. The method of claim 38 wherein the droplets comprise an oil and water emulsion.

40. The method of claim 25, wherein the compartment specific nucleic acid is coupled to a solid substrate, such as with a photo-cleavable DNA spacer.

41. (canceled)

42. (canceled)

43. The method of claim 40, wherein the solid substrate comprises a hydrogel bead.

44. The method of claim 25, wherein the method is used in a functional screening study at a single cell level.

45. The method of claim 25, wherein the population of cells are derived from cell lines.

46. The method of claim 25, wherein the population of cells are primary cells.

47. The method of claim 25, wherein the sample barcode nucleic acid sequence and sequencing adapter are associated with (1) the genomic DNA forward primer or (2) the genomic DNA reverse primer.

48. (canceled)

49. The method of claim 25, wherein the sample barcode nucleic acid sequence, and sequencing adaptor specifically binds to (1) the RTC-PCR forward primer; or (2) the RTC-PCR reverse primer.

50. (canceled)
